(19) United States
(12) Patent Application Publication
(10) Pub. No.: US 2026/0184580 A1
(43) Pub. Date: Jul. 2, 2026

(54) METHOD FOR SYNTHESIZING HIGH-VALUE PRODUCTS FROM REFINERY FURNACE GAS

(71) Applicant: KUNMING UNIVERSITY OF SCIENCE AND TECHNOLOGY, KUNMING (CN)

(72) Inventors: KONGZHAI LI, KUNMING (CN); HUICONG ZUO, KUNMING (CN); HUA WANG, KUNMING (CN); LEI JIANG, KUNMING (CN); DANYANG LI, KUNMING (CN); HAN ZHAO, KUNMING (CN)

(21) Appl. No.: 19/409,674

(22) Filed: Dec. 4, 2025

(30) Foreign Application Priority Data
Dec. 31, 2024 (CN) 202411986496.4

(51) Int. Cl. C01C 1/04; B01D 53/86; C07C 31/04

(52) U.S. Cl. CPC C01C 1/0417; B01D 53/8678; B01D 53/869; C07C 31/04; B01D 2256/10; B01D 2256/22; B01D 2258/02

(57) ABSTRACT

A method for synthesizing high-value products from refinery furnace gas is provided. Crude furnace gas from a refinery is purified, and then clean furnace gas is delivered to a three-stage normal pressure reactor in a chemical looping, where nearly pure carbon dioxide, nitrogen, and hydrogen products are obtained in each stage of the process. Nearly pure hydrogen reformed from coke oven gas is used for hydrogen replenishment to adjust a gas structure. Mixed raw materials that meet standards are pressurized and delivered to a synthesizer, and methanol or ammonia products are obtained after gas-liquid separation.

### CROSS REFERENCE TO RELATED APPLICATIONS

The present disclosure claims the priority to the Chinese patent application with the filing No. 202411986496.4, entitled “METHOD FOR SYNTHESIZING HIGH-VALUE PRODUCTS FROM REFINERY FURNACE GAS” and filed on Dec. 31, 2024 with the Chinese Patent Office, the contents of which are incorporated herein by reference in their entirety.

### TECHNICAL FIELD

The present disclosure relates to the technical field of energy conversion and chemical synthesis, and in particular, to a method for synthesizing high-value products from refinery furnace gas.

### BACKGROUND

The energy consumption of China's steel industry accounts for about 15% of the national total industrial energy consumption, the exhaust emissions account for 26% of the total industrial emissions, and the carbon emissions account for about 15% of the national total carbon emissions. Refineries produce blast furnace gas/converter gas mainly composed of carbon monoxide (CO), carbon dioxide (CO₂), and nitrogen (N₂) over the long term. Refinery furnace gas is the gas generated during iron and steel production processes, specifically the blast furnace gas or converter gas. Refinery furnace gas, after being used for heating by enterprises, is primarily used for power generation, with a thermal efficiency of merely 30% to 40%. The use of refinery furnace gas for power generation (mainly carbon monoxide combustion) has high carbon emissions, with a carbon emission coefficient of 1940 grams of CO₂ equivalent per kilowatt hour, which is much higher than that of China's power grid (590 grams of CO₂ equivalent per kilowatt hour), and even higher than that of coal power generation (930 grams of CO₂ equivalent per kilowatt hour). Currently, furnace gas used by relevant refinery enterprises in China is basically balanced, but whether it is used in the process or for power generation, the carbon monoxide in the furnace gas is converted into carbon dioxide emissions, making it difficult to achieve essential reduction of carbon dioxide emissions.

It is a key link in reducing greenhouse gas emissions from steel and chemical industries to produce high value-added products (such as methanol, ethanol, natural gas, or polymers) via chemical carbonylation from refinery furnace gas as a raw material instead of coal-based carbon monoxide while the conversion of carbon elements in refinery furnace gas into electricity with high carbon emission coefficients is avoided. The extraction of carbon monoxide from the refinery furnace gas as a non-fossil raw material improves the quality of gas utilization and cancels conventional carbon monoxide generating and air separation processes, buts faces the challenge that a large quantity of carbon dioxide and nitrogen resources in furnace gas cannot be utilized and are emitted.

In addition, the synthesis of the above-mentioned typical chemical products generally involves hydrogenation. Enterprises equipped with coking processes can achieve hydrogenation by separating hydrogen from coke oven gas, while enterprises without coking processes first need to solve cheap hydrogen sources. Meanwhile, the synthesis of typical chemical products requires matching raw material structures (such as hydrogen-to-carbon ratio), and usually requires gas replenishment to make up for the process, resulting in some limitations in large-scale promotion and application.

### SUMMARY

The present disclosure aims to provide a method for synthesizing high-value products from refinery furnace gas, to solve the above problems of low utilization of refinery furnace gas and generation of carbon dioxide greenhouse gas.

The refinery furnace gas is produced in conventional iron and steel production processes, with carbon and hydrogen as main components, including blast furnace gas and converter gas. The method uses the furnace gas produced by refineries over the long term as a non-fossil raw material. By optimizing the structures of gas products (hydrogen-carbon ratio and hydrogen-nitrogen ratio) obtained in various stages of a chemical looping reforming three-stage reactor, pressurizing the gas products, and delivering the pressurized gas products to a methanol or ammonia synthesizer, the method solves the problem that carbon dioxide and nitrogen cannot be utilized when high-value products are synthesized by chemical carbonylation from refinery furnace gas instead of coal-based carbon monoxide in the past, and also solves the problems of additional costs and carbon emissions faced by other hydrogen production methods.

The method optimizes the process flow by using an oxygen-depleted air (mainly nitrogen) product obtained in an air reactor of a chemical looping three-stage reactor during oxidation as a high-purity nitrogen source for ammonia synthesis, thereby solving the problem of nitrogen loss caused by long-term emission of the oxygen-depleted air product in conventional processes.

The method also produces synthesis gas (carbon monoxide and hydrogen) and hydrogen from coke oven gas produced in a coke oven process through chemical looping reforming, where the synthesis gas can be returned to a waste heat boiler for heat recovery and used as a product or delivered to the methanol synthesizer after gas structure (hydrogen-carbon ratio) adjustment as needed, while the hydrogen can be used alone for hydrogen replenishment to compensate for the mismatch of raw material structure in the process, thereby expanding the industrial route of refinery furnace gas and solving the problem of cheap hydrogen (0.868 yuan/Nm³) replenishment.

The method optimizes the process flow by purifying excess unreacted gas in the methanol or ammonia synthesizer through an absorption tower to reach a gas structure required for methanol or ammonia synthesis, and then recycling the purified unreacted gas in a mixing homogenization process to replace some fresh gas, thereby solving the problem that the long-term circulation and enrichment of a large amount of unreacted gas requires regular emptying or supplementation of fresh gas to maintain normal process operation due to low single-pass conversion rate of synthesis gas.

In order to overcome the above problems in the prior art, specific technical solutions provided by the present disclosure are as follows:
A method for synthesizing high-value products from refinery furnace gas includes a raw material preparation process and a product preparation process, where
the raw material preparation process includes the following steps:
S1. removing dust and pollutants from crude furnace gas discharged from a refinery unit to obtain clean furnace gas;
S2. recovering heat energy from the clean furnace gas via a first waste heat boiler, then delivering the clean furnace gas to a first fuel reactor filled with a solid oxygen carrier material, oxidizing the clean furnace gas by the solid oxygen carrier material to generate carbon dioxide, outputting carbon dioxide and nitrogen mixed exhaust gas at a tail end of the first fuel reactor, and separating the exhaust gas by adsorption to obtain nearly pure carbon dioxide and nearly pure nitrogen;
S3. delivering an oxygen-depleted solid material transformed from the solid oxygen carrier material after step S2 to a first steam reactor, delivering water vapor to the first steam reactor, and reforming the oxygen-depleted solid material and the water vapor to generate hydrogen, where the oxygen-depleted solid material is subjected to partial oxygen recovery to obtain a partially oxygen-recovered solid material;
S4. delivering the partially oxygen-recovered solid material to an air reactor in which air reacts with the partially oxygen-recovered solid material to obtain nearly pure nitrogen; and
S5. removing dust and pollutants from crude coke oven gas discharged from another refinery unit to obtain clean coke oven gas, delivering the clean coke oven gas to a second fuel reactor filled with a solid oxygen carrier material, oxidizing the clean coke oven gas by the solid oxygen carrier material to obtain synthesis gas including carbon monoxide and hydrogen, delivering an oxygen-depleted solid material transformed from the solid oxygen carrier material to a second steam reactor in a second stage, delivering water vapor to the second steam reactor, and reforming the oxygen-depleted solid material and the water vapor to generate hydrogen;
where the product preparation process includes the following steps:
S6. adjusting a hydrogen-carbon ratio or hydrogen-nitrogen ratio of the nearly pure carbon dioxide, the nearly pure nitrogen, and the nearly pure hydrogen, and delivering the adjusted nearly pure carbon dioxide and hydrogen to a mixing homogenizer to prepare methanol or ammonia synthesis gas;
S7. delivering the methanol or ammonia synthesis gas to a pressure buffer for pressurization, and then delivering the pressurized synthesis gas to a tower pre-heater; and
S8. delivering the pre-heated methanol or ammonia synthesis gas to a synthesis tower for synthesis reaction, and obtaining methanol or ammonia products after gas-liquid separation.

Preferably, in step S1, the clean furnace gas is clean blast furnace gas or clean converter gas.

Preferably, in step S1, the crude furnace gas is delivered to a three-stage fully dry purification bag filter for dust removal, and the dust-removed furnace gas is sequentially delivered to a catalytic purifier and an adsorption purifier to remove pollutants and obtain the clean furnace gas.

Preferably, in step S2, the temperature of the first fuel reactor is set to 550° C. to 650° C., and the solid oxygen carrier material is one of a self-made cerium iron zirconium/Al₂O₃ honeycomb ceramic monolithic oxygen carrier and a self-made cerium iron zirconium/MgO honeycomb ceramic monolithic oxygen carrier, as disclosed in the invention authorization CN101857458B.

Preferably, in step S3, the reaction temperature is 550° C. to 650° C., and the water vapor is generated by a steam generator that is driven by low-grade heat energy recovered by the first waste heat boiler.

Preferably, in step S4, the reaction temperature is 550° C. to 650° C., and the partially oxygen-recovered solid material is subjected to complete oxygen recovery to obtain the solid oxygen carrier material, which is then delivered back to the first fuel reactor for recycling; and heat energy carried by the nearly pure nitrogen is recovered by the first waste heat boiler.

Preferably, in step S5, the temperature of the second fuel reactor is set to 800° C. to 850° C., and the solid oxygen carrier material is one of a self-made A-site nickel-doped perovskite oxygen carrier and a self-made A-site strontium-doped perovskite oxygen carrier, as disclosed in the invention authorization CN111232920B; and the water vapor is generated by the steam generator that is driven by the low-grade heat energy recovered by the first waste heat boiler.

Preferably, in the product preparation process, a self-made ordered-grade porous copper-based catalyst is used as the methanol synthesis catalyst, as disclosed in invention application CN117101665A, and an iron-based catalyst is used as the ammonia synthesis catalyst.

Preferably, the product preparation process includes an in-situ methanol preparation process and an in-situ ammonia preparation process arranged in parallel.

Preferably, the methanol preparation process includes the following steps:
S61. adjusting a hydrogen-carbon ratio of the carbon dioxide from step S2, the hydrogen from step S3, and the hydrogen from step S5 to 3.0, and then delivering the adjusted carbon dioxide and hydrogen to a first mixing homogenizer to prepare the methanol synthesis gas;
S71. delivering the methanol synthesis gas to a first pressure buffer for pressurization to 3 MPa to 5 MPa, and then delivering the pressurized methanol synthesis gas to a first tower pre-heater, where the temperature of the first tower pre-heater is set to 130° C. to 150° C.; and
S81. delivering the pre-heated methanol synthesis gas to a first synthesis tower for synthesis reaction to obtain a reaction product, where the temperature of the first synthesis tower is set to 200° C. to 250° C.; performing gas-liquid separation on the reaction product at the tail end of the first synthesis tower to obtain methanol, introducing the unreacted gas desorbed from the top of the first synthesis tower into a first absorption tower, purifying the unreacted gas in the first absorption tower, then delivering the purified unreacted gas back to the first mixing homogenizer to replace some fresh methanol synthesis gas, and recovering the low-grade heat energy in the depressurized unreacted gas by a second waste heat boiler to the first tower pre-heater to replace some heat energy for use.

Preferably, the ammonia preparation process includes the following steps:
S62. adjusting a hydrogen-nitrogen ratio of the nitrogen from step S2 or S4, the hydrogen from step S3, and the hydrogen from step S5 to 2.7 to 3.0, and then delivering the adjusted nitrogen and hydrogen to a second mixing homogenizer to prepare the ammonia synthesis gas;
S72. delivering the ammonia synthesis gas to a second pressure buffer for pressurization to 7 MPa to 8 MPa, and then delivering the pressurized ammonia synthesis gas to a second tower pre-heater, where the temperature of the second tower pre-heater is set to 150° C. to 200° C.; and
S82. delivering the pre-heated ammonia synthesis gas to a second synthesis tower for synthesis reaction to obtain a reaction product, where the temperature of the second synthesis tower is set to 280° C. to 320° C.; performing gas-liquid separation on the reaction product at the tail end of the second synthesis tower to obtain liquid ammonia, introducing the unreacted gas desorbed from the top of the second synthesis tower into a second absorption tower, purifying the unreacted gas in the second absorption tower, then delivering the purified unreacted gas back to the second mixing homogenizer to replace some fresh ammonia synthesis gas, and recovering the low-grade heat energy in the depressurized unreacted gas by a third waste heat boiler to the second tower pre-heater to replace some heat energy for use.

Compared to the prior art, the method for synthesizing high-value products from refinery furnace gas in the present disclosure has the following beneficial effects:
1. The method of the present disclosure comprehensively and efficiently utilizes various levels of resources in refinery furnace gas, achieving maximum development and application. Through complementary utilization of carbon, hydrogen, and nitrogen contained in the refinery furnace gas emitted over the long term, the method effectively solves the problem of effective utilization of a large amount of carbon dioxide and nitrogen when the refinery furnace gas is used as a raw material to replace coal-based carbon monoxide in the past. The method not only promotes deep integration and utilization of resources, but also achieves near zero carbon dioxide emissions.
2. Since the production process of the method is based on the chemical looping reforming of refinery furnace gas, the inherent high purities of end products avoid interference from other inert gases at the contact layer temperature, and quality improvement is achieved without high investment compared to conventional refinery furnace gas component extraction processes.
3. The method of the present disclosure uses the gas product obtained in the coking process for internal self-replenishment of the system according to demand, avoiding additional hydrogen production investment and energy consumption. When renewable energy develops rapidly in the future, the renewable energy can be incorporated into the network described in the present disclosure without large-scale modifications to the prior art.
4. The method of the present disclosure optimizes the gas recycling process in the production process by recycling low-grade nitrogen-based exhaust gas or unreacted gas for internal use in the system, facilitating separate operation of local process shutdown, greatly reducing long-term operation damage of equipment, retaining most of original production equipment and devices, achieving qualitative changes in comprehensive efficiency and benefit improvements with only appropriate modifications, and facilitating industrial promotion within a short time.
5. The method of the present disclosure optimizes intermittent fluctuations of workloads in the production process, and achieves high-level production of product gas through gas buffer linkage between various production links. The low-grade heat energy recovered by the waste heat boiler is used to replace some high-grade fuel combustion heat supply, alleviating heat consumption in the combustion process and achieving gradient utilization of chemical energy.
6. The method of the present disclosure solves the problem that the current thermal catalytic hydrogenation reaction system and photocatalytic/electrocatalytic reduction reaction system rely on purchased standard gases as raw materials, which is not conducive to the transition of catalytic reactions from laboratory scales to industrial scales, thereby providing a feasible solution for the emission and reuse of complex gas sources throughout the entire life cycle.

### BRIEF DESCRIPTION OF THE DRAWINGS

FIG. 1 is a schematic flow diagram of a raw material preparation process; and

FIG. 2 is a schematic flow diagram of a product preparation process.

Explanation of reference numerals: 1. First refinery unit; 2. Second refinery unit; 3. First three-way valve; 4. Bag filter; 5. Catalytic purifier; 6. First adsorption purifier; 7. First waste heat boiler; 8. Second three-way valve; 9. First pressure reducing valve; 10. First electromagnetic flowmeter; 11. Third three-way valve; 12. First fuel reactor; 13. Adsorption separator; 14. First saturation storage tank; 15. Second saturation storage tank; 16. Steam generator; 17. Fourth three-way valve; 18. Second electromagnetic flowmeter; 19. First steam reactor; 20. First heat exchanger; 21. Fifth three-way valve; 22. Third saturation storage tank; 23. Air reactor; 24. Second adsorption purifier; 25. Sixth three-way valve; 26. Third electromagnetic flowmeter; 27. Second pressure reducing valve; 28. Fourth electromagnetic flowmeter; 29. Second fuel reactor; 30. Second heat exchanger; 31. Fifth electromagnetic flowmeter; 32. Second steam reactor; 33. Third heat exchanger; 34. Fourth saturation storage tank; 35. Third pressure reducing valve; 36. Sixth electromagnetic flowmeter; 37. Fourth pressure reducing valve; 38. Seventh three-way valve; 39. Seventh electromagnetic flowmeter; 40. Fifth pressure reducing valve; 41. Eighth electromagnetic flowmeter; 42. Eighth three-way valve; 43. First mixing homogenizer; 44. First pressure buffer; 45. First pressure gage; 46. Ninth electromagnetic flowmeter; 47. First tower pre-heater; 48. First synthesis tower; 49. Fourth heat exchanger; 50. First gas-liquid separator; 51. First absorption tower; 52. Second waste heat boiler; 53. Tenth electromagnetic flowmeter; 54. Sixth pressure reducing valve; 55. Eleventh electromagnetic flowmeter; 56. Ninth three-way valve; 57. Second mixing homogenizer; 58. Second pressure buffer; 59. Second pressure gage; 60. Twelfth electromagnetic flowmeter; 61. Second tower pre-heater; 62. Second synthesis tower; 63. Fifth heat exchanger; 64. Second gas-liquid separator; 65. Second absorption tower; 66. Third waste heat boiler.

### DETAILED DESCRIPTION

In order to further elaborate the technical means taken by the present disclosure to achieve the intended disclosure objectives and elaborate the achieved effects, the specific implementations, structures, features, and effects of the present disclosure will be described in detail below in conjunction with preferred embodiments.

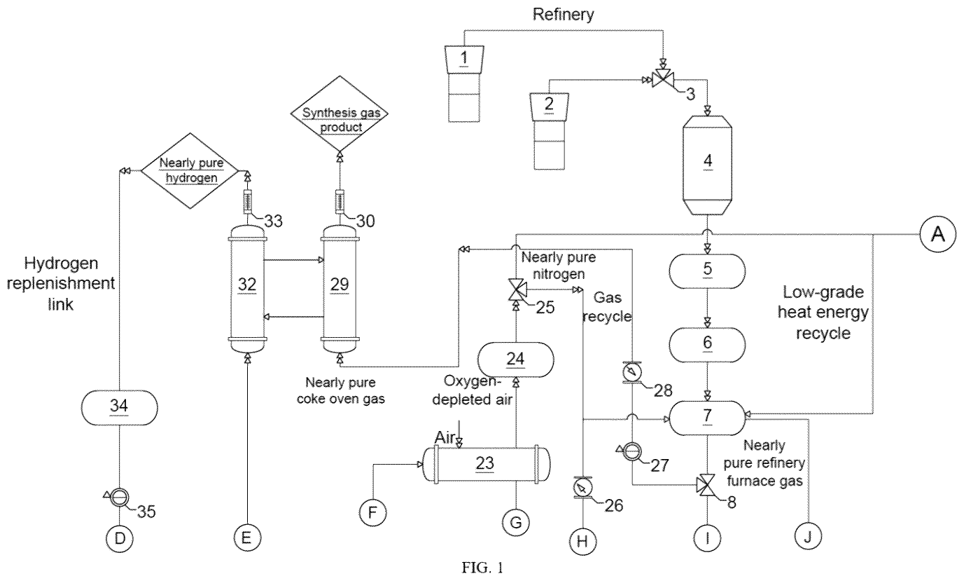

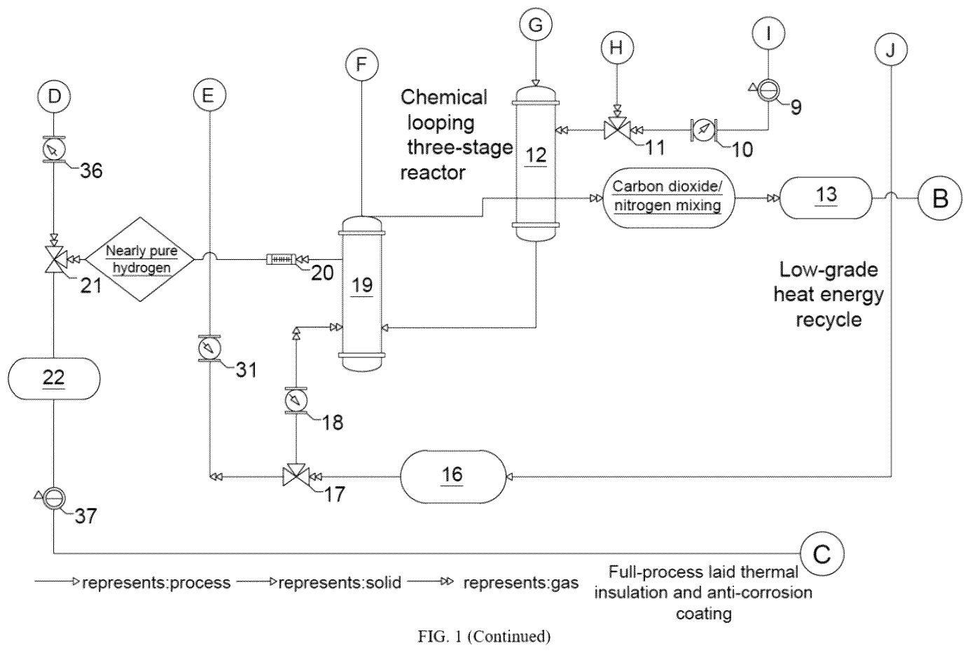

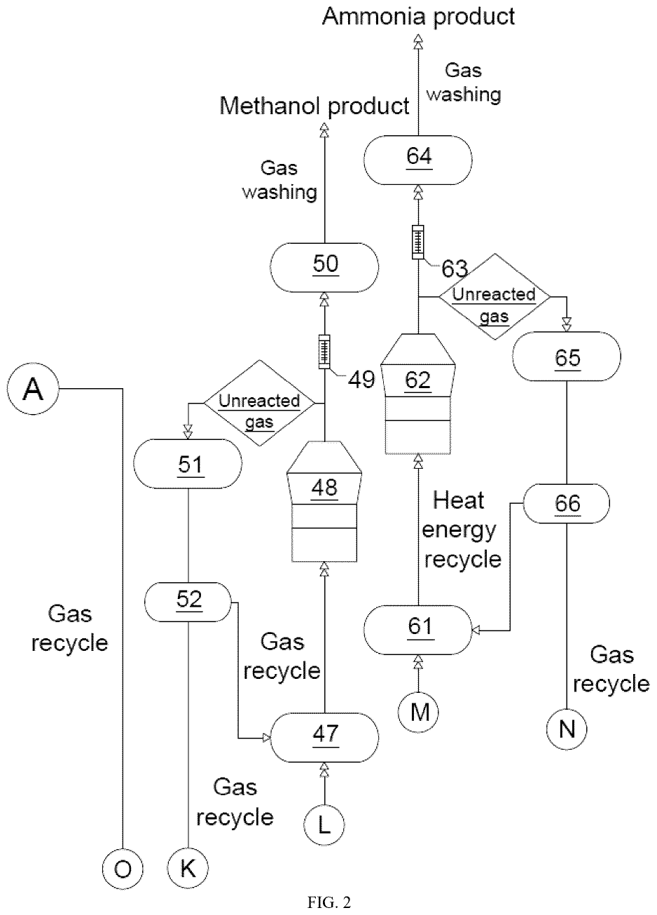

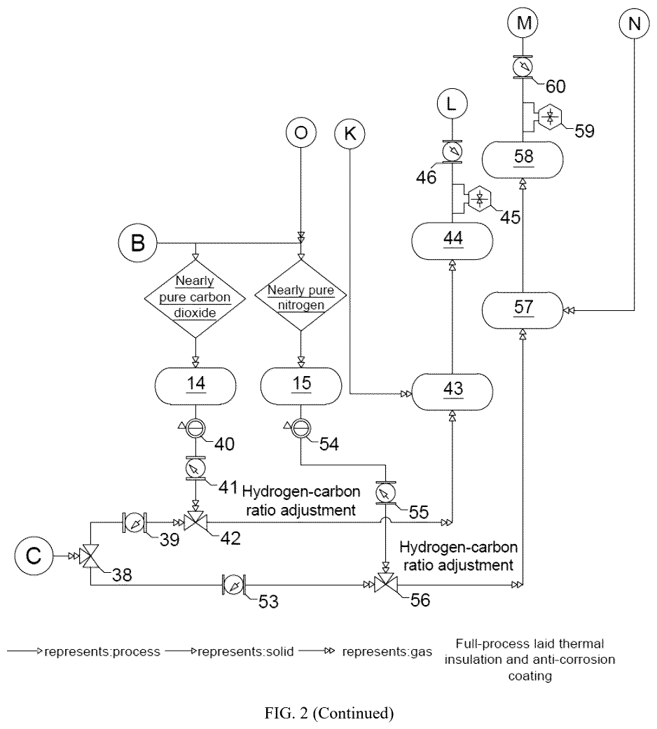

The present disclosure provides a method for synthesizing high-value products from refinery furnace gas. The raw material preparation process of FIG. 1 and the product preparation process of FIG. 2 constitute the entire process of the method for synthesizing high-value products from refinery furnace gas. With reference to FIGS. 1-2, the specific implementation of the present disclosure is as follows:
Crude furnace gas produced by a refinery unit, including crude blast furnace gas and converter gas, is purified by a dust removal and purification unit to obtain clean blast furnace gas or converter gas, where low-grade heat energy in the furnace gas is recovered by a first waste heat boiler 7, the clean blast furnace gas or converter gas is delivered as a gas fuel to a first fuel reactor 12 of a chemical looping three-stage reactor, and exhaust gas containing nitrogen and carbon dioxide that is produced by fuel combustion is introduced into a molecular sieve adsorption separator 13 to obtain nearly pure carbon dioxide and nearly pure nitrogen products, which are then delivered to a first saturation storage tank 14 and a second saturation storage tank 15 respectively.

The low-grade heat energy recovered by the first waste heat boiler 7 is delivered to a steam generator 16, steam generated by the steam generator 16 is delivered to a first steam reactor 19 for reforming reaction to obtain a product, the product is subjected to heat exchange in a first heat exchanger 20 to obtain nearly pure hydrogen, and the hydrogen is delivered to a third saturation storage tank 22 through a fifth three-way valve 21;
Clean air is delivered to an air reactor 23 for oxidation reaction, and the resulting oxygen-depleted air, i.e., nearly pure nitrogen, is reused in the chemical looping system or delivered to the second saturation storage tank 15; and the heat energy generated in the high-temperature oxygen-depleted air is recovered by the first waste heat boiler 7.

Crude coke oven gas produced by a coke oven unit is purified by the dust removal and purification unit to obtain clean coke oven gas, low-grade heat energy in the oven gas is recovered by the first waste heat boiler 7, the clean coke oven gas is delivered as a gas fuel to a second fuel reactor 29, and exhaust gas containing hydrogen and carbon monoxide that is produced by fuel combustion is subjected to heat exchange in a second heat exchanger 30 to obtain a synthesis gas product.

The low-grade heat energy recovered by the first waste heat boiler 7 is delivered to the steam generator 16, steam generated by the steam generator 16 is delivered to a second steam reactor 32 for reforming reaction to obtain a product, and the product is subjected to heat exchange in a third heat exchanger 33 to obtain nearly pure hydrogen, which is then delivered to a fourth saturation storage tank 34.

The carbon dioxide in the first saturation storage tank 14 and the hydrogen in the third saturation storage tank 22 are adjusted for a hydrogen-carbon ratio, where insufficient hydrogen is replenished by the hydrogen in the fourth saturation storage tank 34; the ratio-adjusted gas flow is delivered to a first mixing homogenizer 43 and prepared into methanol synthesis gas in the first mixing homogenizer 43; the methanol synthesis gas, after being pressurized into a standard pressure in a first pressure buffer 44, is delivered to a first tower pre-heater 47, the pre-heated high-pressure methanol synthesis gas is delivered to a first synthesis tower 48, and a desorbed product from the top of the first synthesis tower 48 is delivered to a first gas-liquid separator 50 for gas-liquid separation to obtain a methanol product; the depressurized unreacted gas desorbed from the top of the first synthesis tower 48 is delivered to a first absorption tower 51 for absorption and purification and then delivered back to the first mixing homogenizer 43 to replace some fresh methanol synthesis gas; and low-grade heat energy in the depressurized unreacted gas is recovered by a second waste heat boiler 52 and delivered to the first tower pre-heater 47 to replace some heat energy.

The nitrogen in the second saturation storage tank 15 and the hydrogen in the third saturation storage tank 22 are adjusted for a hydrogen-nitrogen ratio, where insufficient hydrogen is replenished by the hydrogen in the fourth saturation storage tank 34; the ratio-adjusted gas flow is delivered to a second mixing homogenizer 57 and prepared into ammonia synthesis gas in the second mixing homogenizer 57; the ammonia synthesis gas, after being pressurized into a standard pressure in a second pressure buffer 58, is delivered to a second tower pre-heater 61, the pre-heated ammonia synthesis gas is delivered to a second synthesis tower 62, and a desorbed product from the top of the second synthesis tower 62 is delivered to a second gas-liquid separator 64 for gas-liquid separation to obtain a liquid ammonia product; the depressurized unreacted gas desorbed from the top of the second synthesis tower 62 is delivered to a second absorption tower 65 for absorption and purification and then delivered back to the second mixing homogenizer 57 to replace some fresh ammonia synthesis gas; and low-grade heat energy in the depressurized unreacted gas is recovered by a third waste heat boiler 66 and delivered to the second tower pre-heater 61 to replace some heat energy. By optimizing the process design, the third waste heat boiler 66 can be completely replaced by the second waste heat boiler 52, that is, the second waste heat boiler 52 and the third waste heat boiler can be integrated into one waste heat boiler for use.

The method for synthesizing high-value products from refinery furnace gas in the present disclosure is implemented by one of the following devices, as shown in FIGS. 1-2.

The refinery unit includes a first unit and a second unit, a furnace gas outlet of the refinery unit is connected to an inlet of a first three-way valve 3, an outlet of the first three-way valve 3 is connected to an inlet of a bag filter 4, an outlet of the bag filter 4 is connected to an inlet of a catalytic purifier 5, an outlet of the catalytic purifier 5 is connected to an inlet of a first adsorption purifier 6, an outlet of the first adsorption purifier 6 is connected to an inlet of the first waste heat boiler 7, a heat energy outlet of the first waste heat boiler 7 is connected to a heat energy inlet of the steam generator 16, and a gas outlet of the first waste heat boiler 7 serves as an inlet allowing the refinery furnace gas to enter the first fuel reactor 12 in a chemical looping system; a second three-way valve 8, a first pressure reducing valve 9, a first electromagnetic flowmeter 10, and a third three-way valve 11 are provided on a connecting pipeline between the first waste heat boiler 7 and the first fuel reactor 12 in a gas flow direction;
A gas outlet of the first fuel reactor 12 in the chemical looping is connected to a gas inlet of the adsorption separator 13, and the adsorption separator 13 separates carbon dioxide and nitrogen, which are then stored in the first saturation storage tank 14 and the second saturation storage tank 15 respectively.

A gas outlet of the first steam reactor 19 in the chemical looping is connected to a gas inlet of the first heat exchanger 20, and hydrogen is stored in the third saturation storage tank 22 after heat exchange.

A heat energy outlet of the air reactor 23 in the chemical looping is connected to the inlet of the first waste heat boiler 7; a gas outlet of the air reactor 23 is connected to the first fuel reactor 12 and the second saturation storage tank 15 via a sixth three-way valve 25, and a third electromagnetic flowmeter 26 is installed on a connecting pipeline between the sixth three-way valve 25 and the first fuel reactor 12.

The gas outlet of the first waste heat boiler 7 is also connected to a gas inlet of the second fuel reactor 29 via the second three-way valve 8, a second pressure reducing valve 27 and a fourth electromagnetic flowmeter 28 are installed on their connecting pipeline, and the second fuel reactor 29 is used to generate synthesis gas products.

Water vapor generated by the steam generator 16 enters the first steam reactor 19 and the second steam reactor 32 via a fourth three-way valve 17, a second electromagnetic flowmeter 18 is installed on a connecting pipeline between the fourth three-way valve 17 and the first steam reactor 19, a fifth electromagnetic flowmeter 31 is installed on a connecting pipeline between the fourth three-way valve 17 and the second steam reactor 32, a gas outlet of the second steam reactor 32 is connected to a gas inlet of the third heat exchanger 33, and hydrogen is stored in the fourth saturation storage tank 34 after heat exchange;
The third saturation storage tank 22 and the fourth saturation storage tank 34 are connected to each other, and a third pressure reducing valve 35 and a sixth electromagnetic flowmeter 36 are installed on their connecting pipeline.

The third saturation storage tank 22 is connected to the first mixing homogenizer 43 and the second mixing homogenizer 57 of a synthesis system via a seventh three-way valve 38, a fourth pressure reducing valve 37 is installed on a connecting pipeline between the third saturation storage tank 22 and the seventh three-way valve 38, and a seventh electromagnetic flowmeter 39 is installed on a connecting pipeline between the seventh three-way valve 38 and the first mixing homogenizer 43; a gas inlet of the first mixing homogenizer 43 is also connected to the first saturation storage tank 14, and a fifth pressure reducing valve 40, an eighth electromagnetic flowmeter 41, and an eighth three-way valve 42 are installed on their connecting pipeline; a gas outlet of the first mixing homogenizer 43 is connected to a gas inlet of the first pressure buffer 44; a gas outlet of the first pressure buffer 44 is connected to a gas inlet of the first tower pre-heater 47, and a first pressure gage 45 and a ninth electromagnetic flowmeter 46 are installed on their connecting pipeline; a gas outlet of the first tower pre-heater 47 is connected to a gas inlet of the first synthesis tower 48, the first synthesis tower 48 is used to synthesize methanol products, a gas outlet of the first synthesis tower 48 is connected to a fourth heat exchanger 49 and then to an inlet of the first gas-liquid separator 50, an unreacted top gas outlet of the first synthesis tower 48 is connected to an inlet of the first absorption tower 51, an outlet of the first absorption tower 51 is connected to an inlet of the second waste heat boiler 52, a heat energy outlet of the second waste heat boiler 52 is connected to an inlet of the first tower pre-heater 47, a gas outlet of the second waste heat boiler 52 is connected to an inlet of the first mixing homogenizer 43, and an outlet product from the first gas-liquid separator 50 serves as a final product of the present disclosure.

A tenth electromagnetic flowmeter 53 is installed on a connecting pipeline between the seventh three-way valve 38 and the second mixing homogenizer 57; a gas inlet of the second mixing homogenizer 57 is also connected to the second saturation storage tank 15, and a sixth pressure reducing valve 54, an eleventh electromagnetic flowmeter 55, and a ninth three-way valve 56 are installed on their connecting pipeline; a gas outlet of the second mixing homogenizer 57 is connected to a gas inlet of the second pressure buffer 58; a gas outlet of the second pressure buffer 58 is connected to a gas inlet of the second tower pre-heater 61, and a second pressure gage 59 and a twelfth electromagnetic flowmeter 60 are installed on their connecting pipeline; a gas outlet of the second tower pre-heater 61 is connected to a gas inlet of the second synthesis tower 62, the second synthesis tower 62 is used to synthesize ammonia products, a gas outlet of the second synthesis tower 62 is connected to a fifth heat exchanger 63 and then to an inlet of the second gas-liquid separator 64, an unreacted top gas outlet of the second synthesis tower 62 is connected to an inlet of the second absorption tower 65, an outlet of the second absorption tower 65 is connected to an inlet of the third waste heat boiler 66, a heat energy outlet of the third waste heat boiler 66 is connected to an inlet of the second tower pre-heater 61, a gas outlet of the third waste heat boiler 66 is connected to an inlet of the second mixing homogenizer 57, and an outlet product from the second gas-liquid separator 64 serves as a final product of the present disclosure.

In order to better understand the present disclosure, the following embodiments further illustrate the present disclosure, but should not be construed as limiting the present disclosure. Non-essential improvements and adjustments made by those skilled in the art based on the above invention content are also considered to fall within the scope of protection of the present disclosure.

### Embodiment 1

This embodiment provides a method for synthesizing high-value products from refinery furnace gas, including the following steps:
S1. Crude furnace gas discharged from the first refinery unit 1 is delivered to the three-stage fully dry purification bag filter 4 for dust removal to avoid the impact of dust on the quality of furnace gas; and the dust-removed furnace gas is sequentially delivered to the catalytic purifier 5 containing a specific catalyst and the first adsorption purifier 6 containing a specific adsorbent to remove most pollutants and obtain clean blast furnace gas. The specific process may be referred to existing dust removal and purification processes, and will not be explained in detail in Embodiment 1 of the present disclosure.
S2. The clean blast furnace gas that meets dust removal and purification standards is subjected to heat energy recovery by the first waste heat boiler 7 and then delivered to the first fuel reactor 12 in the first stage of the chemical looping three-stage reactor, where the first waste heat boiler 7 is arranged downstream of a purification process, and the recovered furnace gas waste heat is supplied to the subsequent process as low-grade heat energy; the temperature of the first fuel reactor 12 is set to 550° C., and the first fuel reactor 12 is filled with a solid oxygen carrier material, where the solid oxygen carrier material is a self-made cerium iron zirconium/MgO honeycomb ceramic monolithic oxygen carrier; the solid oxygen carrier material loses oxygen in a high temperature environment and undergoes oxidation reaction with carbon monoxide in the blast furnace gas to generate carbon dioxide, and carbon dioxide and nitrogen mixed exhaust gas is output at the tail end of the first fuel reactor 12, where the purity of carbon dioxide is 45%, and the purity of nitrogen is 55%; the carbon dioxide and nitrogen mixed exhaust gas is introduced into the adsorption separator 13 to separate carbon dioxide and nitrogen by an existing adsorption separation process and obtain nearly pure carbon dioxide and nearly pure nitrogen, the nearly pure carbon dioxide is delivered to the first saturation storage tank 14, and the nearly pure nitrogen is delivered to the second saturation storage tank 15. The solid oxygen carrier material used in the first fuel reactor 12 is a self-made cerium iron zirconium honeycomb ceramic monolithic oxygen carrier, as disclosed in the invention authorization CN101857458B.
S3. The solid oxygen carrier material is transformed into an oxygen-depleted solid material after step S2, and the oxygen-depleted solid material is delivered to the first steam reactor 19 in the second stage of the chemical looping three-stage reactor, where the temperature of the first steam reactor 19 is set to 550° C. Meanwhile, the low-grade heat energy recovered by the first waste heat boiler 7 drives the steam generator 16 to generate water vapor, which is then delivered to the first steam reactor 19. The oxygen-depleted solid material and the water vapor undergo reforming reaction in a high temperature environment to generate hydrogen, and the hydrogen is condensed in the first heat exchanger 20 at the tail end of the first steam reactor 19 to output nearly pure hydrogen, which is then delivered to the third saturation storage tank 22, where the purity of hydrogen is 99.9%. The oxygen-depleted solid material is subjected to partial oxygen recovery in the first steam reactor 19 to obtain a partially oxygen-recovered solid material.

S4. The partially oxygen-recovered solid material is delivered to the air reactor 23 in the third stage of the chemical looping three-stage reactor, where the temperature of the air reactor 23 is set to 550° C. Pure air loses oxygen in a high temperature environment, exhaust gas containing a trace amount of unreacted oxygen and nitrogen is output at the tail end of the air reactor 23, the exhaust gas is introduced into the second adsorption purifier 24, and nearly pure nitrogen is enriched by an existing adsorption purification process and delivered to the second saturation storage tank 15, where the purity of nitrogen is 99.9%. The partially oxygen-recovered solid material is subjected to complete oxygen recovery in the air reactor 23, and the completely oxygen-recovered solid material is delivered back to the first fuel reactor 12 in the first stage of the chemical looping three-stage reactor for recycling. Heat energy generated from the high-temperature oxygen-depleted air is recovered by the first waste heat boiler 7.
S5. Crude coke oven gas discharged from the second refinery unit 2 is subjected to dust and pollutant removal to obtain clean coke oven gas, and the coke oven gas that meets dust removal and purification standards is delivered to the second fuel reactor 29 in the first stage of the chemical looping two-stage reactor, where the temperature of the second fuel reactor 29 is set to 850° C., the second fuel reactor 29 is filled with a solid oxygen carrier material, and the solid oxygen carrier material is a self-made A-site strontium-doped perovskite oxygen carrier. The coke oven gas is condensed in the second heat exchanger 30 at the tail end of the second fuel reactor 29 to output a carbon monoxide and hydrogen mixed synthesis gas product, where the synthesis gas product includes carbon monoxide and hydrogen; the synthesis gas may be returned to the first waste heat boiler 7 for heat recovery and then sold as a product, or the synthesis gas may be delivered to a methanol synthesis process after the ratio of carbon monoxide to hydrogen is adjusted as needed;

The solid oxygen carrier material is transformed into an oxygen-depleted solid material, and the oxygen-depleted solid material is delivered to the second steam reactor 32 in the second stage of the chemical looping two-stage reactor. Meanwhile, water vapor generated by the steam generator 16 is delivered to the second steam reactor 32. The oxygen-depleted solid material and the water vapor undergo reforming reaction in a high temperature environment to generate hydrogen, and the hydrogen is condensed in the third heat exchanger 33 at the tail end of the second steam reactor 32 to output nearly pure hydrogen, which is then delivered to the fourth saturation storage tank 34, where the purity of hydrogen is 99.9%. Similarly, the heat energy in the second steam reactor 32 comes from the low-grade heat energy recovered by the first waste heat boiler 7 and drives the steam generator 16 to generate the water vapor. The solid oxygen carrier material used in the second fuel reactor 29 is a self-made A-site doped perovskite oxygen carrier, as disclosed in the invention authorization CN111232920B.
S6. The hydrogen-carbon ratio of the carbon dioxide in the first saturation storage tank 14 in step S2, the hydrogen in the third saturation storage tank 22 in step S3, and the hydrogen in the fourth saturation storage tank 34 in step S5 is adjusted to 3.0, and then the carbon dioxide and the hydrogen are delivered to the first mixing homogenizer 43 to prepare methanol synthesis gas, where the volume percentage of the hydrogen in step S3 accounts for 30%, and the volume percentage of the hydrogen in step S5 accounts for 70%; where the volume percentages of the hydrogen in steps S3 and S5 are affected by respective production capacities.
S7. The methanol synthesis gas is delivered to the first pressure buffer 44, pressurized to 4 MPa by a compressor, and then delivered to the first tower pre-heater 47, where the temperature of the first tower pre-heater 47 is set to 140° C., and the gas buffer linkage is designed in the pressurization process to prevent intermittent fluctuations of workloads in the compression pipeline, thereby providing an excellent working environment for the subsequent methanol synthesis process.
S8. The pre-heated methanol synthesis gas is delivered to the first synthesis tower 48 for reaction with a catalyst to obtain a reaction product, where the catalyst is a self-made 240 nm ordered-grade porous copper-based oxide material, and the temperature of the first synthesis tower 48 is set to 220° C.; the reaction product is condensed in the fourth heat exchanger 49 at the tail end of the first synthesis tower 48 and then introduced into the first gas-liquid separator 50 for gas-liquid separation to obtain crude methanol, achieving a carbon dioxide conversion rate of 14.6% and a methanol selectivity of 71.3%; the unreacted gas desorbed from the top of the first synthesis tower 48 is introduced into the first absorption tower 51, purified in the first absorption tower 51, and then delivered back to the first mixing homogenizer 43 to replace some fresh methanol synthesis gas, where the available volume percentage of the unreacted gas that replaces some fresh methanol synthesis gas is 30%; the second waste heat boiler 52 is arranged downstream of the first absorption tower 51, and the low-grade heat energy in the depressurized unreacted gas is recovered by the second waste heat boiler 52 and delivered to the first tower pre-heater 47 to replace some heat energy for use. The methanol synthesis catalyst is a self-made ordered-grade porous copper-based catalyst, as disclosed in the invention application CN117101665A.

### Embodiment 2

This embodiment provides a method for synthesizing high-value products from refinery furnace gas, including the following steps:
S1. Dust and pollutants are removed from crude furnace gas discharged from the first refinery unit 1 to obtain clean converter gas. The specific process may be referred to existing dust removal and purification processes, and will not be explained in detail in Embodiment 2 of the present disclosure.
S2. The clean converter gas that meets dust removal and purification standards is subjected to heat energy recovery by the first waste heat boiler 7 and then delivered to the first fuel reactor 12 in the first stage of the chemical looping three-stage reactor, where the temperature of the first fuel reactor 12 is set to 650° C., and the first fuel reactor 12 is filled with a solid oxygen carrier material, where the solid oxygen carrier material is a self-made cerium iron zirconium/Al₂O₃ honeycomb ceramic monolithic oxygen carrier; the solid oxygen carrier material loses oxygen in a high temperature environment and undergoes oxidation reaction with carbon monoxide in the converter gas to generate carbon dioxide, and carbon dioxide and nitrogen mixed exhaust gas is output at the tail end of the first fuel reactor 12, where the purity of carbon dioxide is 80%, and the purity of nitrogen is 20%; the carbon dioxide and the nitrogen are separated by an existing adsorption separation process to obtain nearly pure carbon dioxide and nearly pure nitrogen, the nearly pure carbon dioxide is delivered to the first saturation storage tank 14, and the nearly pure nitrogen is delivered to the second saturation storage tank 15.
S3. The solid oxygen carrier material is transformed into an oxygen-depleted solid material after step S2, and the oxygen-depleted solid material is delivered to the first steam reactor 19 in the second stage of the chemical looping three-stage reactor, where the temperature of the first steam reactor 19 is set to 650° C. Meanwhile, the low-grade heat energy recovered by the first waste heat boiler 7 drives the steam generator 16 to generate water vapor, which is then delivered to the first steam reactor 19. The oxygen-depleted solid material and the water vapor undergo reforming reaction in a high temperature environment to generate hydrogen, and the hydrogen is condensed at the tail end of the first steam reactor 19 to output nearly pure hydrogen, which is then delivered to the third saturation storage tank 22, where the purity of hydrogen is 99.9%. The oxygen-depleted solid material is subjected to partial oxygen recovery in the first steam reactor 19 to obtain a partially oxygen-recovered solid material.
S4. The partially oxygen-recovered solid material is delivered to the air reactor 23 in the third stage of the chemical looping three-stage reactor, where the temperature of the air reactor 23 is set to 650° C. Pure air loses oxygen in a high temperature environment, exhaust gas containing a trace amount of unreacted oxygen and nitrogen is output at the tail end of the air reactor 23, and nearly pure nitrogen is enriched by an existing adsorption purification process and delivered to the second saturation storage tank 15, where the purity of nitrogen is 99.9%. The partially oxygen-recovered solid material is subjected to complete oxygen recovery in the air reactor 23, and the completely oxygen-recovered solid material is delivered back to the first fuel reactor 12 in the first stage of the chemical looping three-stage reactor for recycling. Heat energy generated from the high-temperature oxygen-depleted air is recovered by the first waste heat boiler 7.
S5. Crude coke oven gas discharged from the second refinery unit 2 is subjected to dust and pollutant removal to obtain clean coke oven gas, and the coke oven gas that meets dust removal and purification standards is delivered to the second fuel reactor 29 in the first stage of the chemical looping two-stage reactor, where the temperature of the second fuel reactor 29 is set to 800° C., the second fuel reactor 29 is filled with a solid oxygen carrier material, and the solid oxygen carrier material is a self-made A-site nickel-doped perovskite oxygen carrier; a carbon monoxide and hydrogen mixed synthesis gas product is output at the tail end of the second fuel reactor 29, where the synthesis gas product includes carbon monoxide and hydrogen;

The solid oxygen carrier material is transformed into an oxygen-depleted solid material, and the oxygen-depleted solid material is delivered to the second steam reactor 32 in the second stage of the chemical looping two-stage reactor. Meanwhile, water vapor generated by the steam generator 16 is delivered to the second steam reactor 32, the oxygen-depleted solid material and the water vapor undergo reforming reaction in a high temperature environment to generate hydrogen, and the hydrogen is condensed at the tail end of the second steam reactor 32 to output nearly pure hydrogen, which is then delivered to the fourth saturation storage tank 34, where the purity of hydrogen is 99.9%. Similarly, the heat energy in the second steam reactor 32 comes from the low-grade heat energy recovered by the first waste heat boiler 7 and drives the steam generator 16 to generate the water vapor.
S6. The hydrogen-carbon ratio of the carbon dioxide in the first saturation storage tank 14 in step S2, the hydrogen in the third saturation storage tank 22 in step S3, and the hydrogen in the fourth saturation storage tank 34 in step S5 is adjusted to 3.0, and then the carbon dioxide and the hydrogen are delivered to the first mixing homogenizer 43 to prepare methanol synthesis gas, where the volume percentage of the hydrogen in step S3 accounts for 40%, and the volume percentage of the hydrogen in step S5 accounts for 60%; where the volume percentages of the hydrogen in steps S3 and S5 are affected by respective production capacities.
S7. The methanol synthesis gas is delivered to the first pressure buffer 44, pressurized to 4 MPa by a compressor, and then delivered to the first tower pre-heater 47, where the temperature of the first tower pre-heater 47 is set to 140° C., and the buffer prevents intermittent fluctuations of workloads in the compression pipeline, thereby providing an excellent working environment for the subsequent methanol synthesis process.
S8. The pre-heated methanol synthesis gas is delivered to the first synthesis tower 48 for reaction with a catalyst to obtain a reaction product, where the catalyst is a self-made 55 nm ordered-grade porous copper-based oxide material, and the temperature of the first synthesis tower 48 is set to 220° C.; the reaction product is subjected to gas-liquid separation at the tail end of the first synthesis tower 48 to obtain crude methanol, achieving a carbon dioxide conversion rate of 14.9% and a methanol selectivity of 81.0%; the unreacted gas desorbed from the top of the first synthesis tower 48 is introduced into the first absorption tower 51, purified in the first absorption tower 51, and then delivered back to the first mixing homogenizer 43 to replace some fresh methanol synthesis gas, where the available volume percentage of the unreacted gas that replaces some fresh methanol synthesis gas is 27%; and the low-grade heat energy in the depressurized unreacted gas is recovered by the second waste heat boiler 52 and delivered to the first tower pre-heater 47 to replace some heat energy for use.

### Embodiment 3

This embodiment provides a method for synthesizing high-value products from refinery furnace gas, including the following steps:
S1. Dust and pollutants are removed from crude furnace gas discharged from the first refinery unit 1 to obtain clean blast furnace gas. The specific process may be referred to existing dust removal and purification processes, and will not be explained in detail in Embodiment 3 of the present disclosure.
S2. The clean blast furnace gas that meets dust removal and purification standards is subjected to heat energy recovery by the first waste heat boiler 7 and then delivered to the first fuel reactor 12 in the first stage of the chemical looping three-stage reactor, where the temperature of the first fuel reactor 12 is set to 650° C., and the first fuel reactor 12 is filled with a solid oxygen carrier material, where the solid oxygen carrier material is a self-made cerium iron zirconium/Al₂O₃ honeycomb ceramic monolithic oxygen carrier; the solid oxygen carrier material loses oxygen in a high temperature environment and undergoes oxidation reaction with carbon monoxide in the converter gas to generate carbon dioxide, and carbon dioxide and nitrogen mixed exhaust gas is output at the tail end of the first fuel reactor 12, where the purity of carbon dioxide is 80%, and the purity of nitrogen is 20%; the carbon dioxide and the nitrogen are separated by an existing adsorption separation process to obtain nearly pure carbon dioxide and nearly pure nitrogen, the nearly pure carbon dioxide is delivered to the first saturation storage tank 14, and the nearly pure nitrogen is delivered to the second saturation storage tank 15.
S3. The solid oxygen carrier material is transformed into an oxygen-depleted solid material after step S2, and the oxygen-depleted solid material is delivered to the first steam reactor 19 in the second stage of the chemical looping three-stage reactor, where the temperature of the first steam reactor 19 is set to 650° C. Meanwhile, the low-grade heat energy recovered by the first waste heat boiler 7 drives the steam generator 16 to generate water vapor, which is then delivered to the first steam reactor 19. The oxygen-depleted solid material and the water vapor undergo reforming reaction in a high temperature environment to generate hydrogen, and the hydrogen is condensed at the tail end of the first steam reactor 19 to output nearly pure hydrogen, which is then delivered to the third saturation storage tank 22, where the purity of hydrogen is 99.9%. The oxygen-depleted solid material is subjected to partial oxygen recovery in the first steam reactor 19 to obtain a partially oxygen-recovered solid material.
S4. The partially oxygen-recovered solid material is delivered to the air reactor 23 in the third stage of the chemical looping three-stage reactor, where the temperature of the air reactor 23 is set to 650° C. Pure air loses oxygen in a high temperature environment, exhaust gas containing a trace amount of unreacted oxygen and nitrogen is output at the tail end of the air reactor 23, and nearly pure nitrogen is enriched by an existing adsorption purification process and delivered to the second saturation storage tank 15, where the purity of nitrogen is 99.9%. The partially oxygen-recovered solid material is subjected to complete oxygen recovery in the air reactor 23, and the completely oxygen-recovered solid material is delivered back to the first fuel reactor 12 in the first stage of the chemical looping three-stage reactor for recycling. Heat energy generated from the high-temperature oxygen-depleted air is recovered by the first waste heat boiler 7.
S5. Crude coke oven gas discharged from the second refinery unit 2 is subjected to dust and pollutant removal to obtain clean coke oven gas, and the coke oven gas that meets dust removal and purification standards is delivered to the second fuel reactor 29 in the first stage of the chemical looping two-stage reactor, where the temperature of the second fuel reactor 29 is set to 800° C., the second fuel reactor 29 is filled with a solid oxygen carrier material, and the solid oxygen carrier material is a self-made A-site nickel-doped perovskite oxygen carrier; a carbon monoxide and hydrogen mixed synthesis gas product is output at the tail end of the second fuel reactor 29, where the synthesis gas product includes carbon monoxide and hydrogen;

The solid oxygen carrier material is transformed into an oxygen-depleted solid material, and the oxygen-depleted solid material is delivered to the second steam reactor 32 in the second stage of the chemical looping two-stage reactor. Meanwhile, water vapor generated by the steam generator 16 is delivered to the second steam reactor 32, the oxygen-depleted solid material and the water vapor undergo reforming reaction in a high temperature environment to generate hydrogen, and the hydrogen is condensed at the tail end of the second steam reactor 32 to output nearly pure hydrogen, which is then delivered to the fourth saturation storage tank 34, where the purity of hydrogen is 99.9%. Similarly, the heat energy in the second steam reactor 32 comes from the low-grade heat energy recovered by the first waste heat boiler 7 and drives the steam generator 16 to generate the water vapor.
S6. The hydrogen-carbon ratio of the nitrogen in the second saturation storage tank 15 in step S2 or S4, the hydrogen in the third saturation storage tank 22 in step S3, and the hydrogen in the fourth saturation storage tank 34 in step S5 is adjusted to 2.8, and then the nitrogen and the hydrogen are delivered to the second mixing homogenizer 57 to prepare ammonia synthesis gas, where the volume percentage of the hydrogen in step S3 accounts for 40%, and the volume percentage of the hydrogen in step S5 accounts for 60%; where the volume percentages of the hydrogen in steps S3 and S5 are affected by respective production capacities.
S7. The ammonia synthesis gas is delivered to the second pressure buffer 58, pressurized to 7.5 MPa by a compressor, and then delivered to the second tower pre-heater 61, where the temperature of the second tower pre-heater 61 is set to 180° C., and the buffer prevents intermittent fluctuations of workloads in the compression pipeline, thereby providing an excellent working environment for the subsequent ammonia synthesis process.
S8. The pre-heated ammonia synthesis gas is delivered to the second synthesis tower 62 for reaction with a catalyst to obtain a reaction product, where the catalyst is a commercial iron cobalt catalyst, and the temperature of the second synthesis tower 62 is set to 300° C.; the reaction product is condensed in the fifth heat exchanger 63 at the tail end of the second synthesis tower 62 and then delivered to the second gas-liquid separator 64 for gas-liquid separation to obtain crude liquid ammonia, achieving an ammonia volume percentage of 25% in the balanced mixture; the unreacted gas desorbed from the top of the second synthesis tower 62 is introduced into the second absorption tower 65, purified in the second absorption tower 65, and then delivered back to the second mixing homogenizer 57 to replace some fresh ammonia synthesis gas, where the available volume percentage of the unreacted gas that replaces some fresh ammonia synthesis gas is 20%; and the low-grade heat energy in the depressurized unreacted gas is recovered by the third waste heat boiler 66 and delivered to the second tower pre-heater 61 to replace some heat energy for use.

The above describes merely the preferred embodiments of the present disclosure and does not limit the present disclosure in any form. Although the present disclosure is disclosed in the preferred embodiments, the present disclosure is not limited thereto. Any person skilled in the art can use the disclosed technical content to make slight changes or modifications to equivalent embodiments without departing from the scope of the technical solution of the present disclosure. Any brief modifications and equivalent changes made to the above embodiments based on the technical essence of the present disclosure without departing from the technical solution of the present disclosure still fall within the scope of the technical solution of the present disclosure.

What is claimed is:

1. A method for synthesizing high-value products from refinery furnace gas, comprising a raw material preparation process and a product preparation process, wherein
the raw material preparation process comprises the following steps:
S1. removing dust and pollutants from crude furnace gas discharged from a refinery unit to obtain clean furnace gas;
S2. recovering heat energy from the clean furnace gas via a first waste heat boiler, then delivering the clean furnace gas to a first fuel reactor filled with a solid oxygen carrier material, oxidizing the clean furnace gas by the solid oxygen carrier material to generate carbon dioxide, outputting carbon dioxide and nitrogen mixed exhaust gas at a tail end of the first fuel reactor, and separating the exhaust gas by adsorption to obtain nearly pure carbon dioxide and nearly pure nitrogen;
S3. delivering an oxygen-depleted solid material transformed from the solid oxygen carrier material after step S2 to a first steam reactor, delivering water vapor to the first steam reactor, and reforming the oxygen-depleted solid material and the water vapor to generate hydrogen, wherein the oxygen-depleted solid material is subjected to partial oxygen recovery to obtain a partially oxygen-recovered solid material;
S4. delivering the partially oxygen-recovered solid material to an air reactor in which air reacts with the partially oxygen-recovered solid material to obtain nearly pure nitrogen; and
S5. removing dust and pollutants from crude coke oven gas discharged from another refinery unit to obtain clean coke oven gas, delivering the clean coke oven gas to a second fuel reactor filled with a solid oxygen carrier material, oxidizing the clean coke oven gas by the solid oxygen carrier material to obtain synthesis gas comprising carbon monoxide and hydrogen, delivering an oxygen-depleted solid material transformed from the solid oxygen carrier material to a second steam reactor in a second stage, delivering water vapor to the second steam reactor, and reforming the oxygen-depleted solid material and the water vapor to generate hydrogen;
wherein the product preparation process comprises the following steps:
S6. adjusting a hydrogen-carbon ratio or hydrogen-nitrogen ratio of the nearly pure carbon dioxide, the nearly pure nitrogen, and the nearly pure hydrogen, and delivering the adjusted nearly pure carbon dioxide and hydrogen to a mixing homogenizer to prepare methanol or ammonia synthesis gas;
S7. delivering the methanol or ammonia synthesis gas to a pressure buffer for pressurization, and then delivering the pressurized synthesis gas to a tower pre-heater; and
S8. delivering the pre-heated methanol or ammonia synthesis gas to a synthesis tower for synthesis reaction, and obtaining methanol or ammonia products after gas-liquid separation.

2. The method for synthesizing high-value products from refinery furnace gas according to claim 1, wherein in step S1, the crude furnace gas is delivered to a three-stage fully dry purification bag filter for dust removal, and the dust-removed furnace gas is sequentially delivered to a catalytic purifier and an adsorption purifier to remove pollutants and obtain the clean furnace gas.

3. The method for synthesizing high-value products from refinery furnace gas according to claim 1, wherein in step S2, the temperature of the first fuel reactor is set to 550° C. to 650° C., and the solid oxygen carrier material is one of a self-made cerium iron zirconium/Al₂O₃ honeycomb ceramic monolithic oxygen carrier and a self-made cerium iron zirconium/MgO honeycomb ceramic monolithic oxygen carrier.

4. The method for synthesizing high-value products from refinery furnace gas according to claim 1, wherein in step S3, the reaction temperature is 550° C. to 650° C., and the water vapor is generated by a steam generator that is driven by low-grade heat energy recovered by the first waste heat boiler.

5. The method for synthesizing high-value products from refinery furnace gas according to claim 1, wherein in step S4, the reaction temperature is 550° C. to 650° C., and the partially oxygen-recovered solid material is subjected to complete oxygen recovery to obtain the solid oxygen carrier material, which is then delivered back to the first fuel reactor for recycling; and heat energy carried by the nearly pure nitrogen is recovered by the first waste heat boiler.

6. The method for synthesizing high-value products from refinery furnace gas according to claim 1, wherein in step S5, the temperature of the second fuel reactor is set to 800° C. to 850° C., and the solid oxygen carrier material is one of a self-made A-site nickel-doped perovskite oxygen carrier and a self-made A-site strontium-doped perovskite oxygen carrier; and the water vapor is generated by the steam generator that is driven by the low-grade heat energy recovered by the first waste heat boiler.

7. The method for synthesizing high-value products from refinery furnace gas according to claim 1, wherein in the product preparation process, a self-made ordered-grade porous copper-based catalyst is used as the methanol synthesis catalyst, and an iron-based catalyst is used as the ammonia synthesis catalyst.

8. The method for synthesizing high-value products from refinery furnace gas according to claim 1, wherein the product preparation process comprises an in-situ methanol preparation process and an in-situ ammonia preparation process arranged in parallel.

9. The method for synthesizing high-value products from refinery furnace gas according to claim 8, wherein the methanol preparation process comprises the following steps:
S61. adjusting a hydrogen-carbon ratio of the carbon dioxide from step S2, the hydrogen from step S3, and the hydrogen from step S5 to 3.0, and then delivering the adjusted carbon dioxide and hydrogen to a first mixing homogenizer to prepare the methanol synthesis gas;
S71. delivering the methanol synthesis gas to a first pressure buffer for pressurization to 3 MPa to 5 MPa, and then delivering the pressurized methanol synthesis gas to a first tower pre-heater, wherein the temperature of the first tower pre-heater is set to 130° C. to 150° C.; and
S81. delivering the pre-heated methanol synthesis gas to a first synthesis tower for synthesis reaction to obtain a reaction product, wherein the temperature of the first synthesis tower is set to 200° C. to 250° C.; performing gas-liquid separation on the reaction product at the tail end of the first synthesis tower to obtain methanol, introducing the unreacted gas desorbed from the top of the first synthesis tower into a first absorption tower, purifying the unreacted gas in the first absorption tower, then delivering the purified unreacted gas back to the first mixing homogenizer to replace some fresh methanol synthesis gas, and recovering the low-grade heat energy in the depressurized unreacted gas by a second waste heat boiler to the first tower pre-heater to replace some heat energy for use.

10. The method for synthesizing high-value products from refinery furnace gas according to claim 8, wherein the ammonia preparation process comprises the following steps:
S62. adjusting a hydrogen-nitrogen ratio of the nitrogen from step S2 or S4, the hydrogen from step S3, and the hydrogen from step S5 to 2.7 to 3.0, and then delivering the adjusted nitrogen and hydrogen to a second mixing homogenizer to prepare the ammonia synthesis gas;
S72. delivering the ammonia synthesis gas to a second pressure buffer for pressurization to 7 MPa to 8 MPa, and then delivering the pressurized ammonia synthesis gas to a second tower pre-heater, wherein the temperature of the second tower pre-heater is set to 150° C. to 200° C.; and
S82. delivering the pre-heated ammonia synthesis gas to a second synthesis tower for synthesis reaction to obtain a reaction product, wherein the temperature of the second synthesis tower is set to 280° C. to 320° C.; performing gas-liquid separation on the reaction product at the tail end of the second synthesis tower to obtain liquid ammonia, introducing the unreacted gas desorbed from the top of the second synthesis tower into a second absorption tower, purifying the unreacted gas in the second absorption tower, then delivering the purified unreacted gas back to the second mixing homogenizer to replace some fresh ammonia synthesis gas, and recovering the low-grade heat energy in the depressurized unreacted gas by a third waste heat boiler to the second tower pre-heater to replace some heat energy for use.
